(19) United States
(12) Patent Application Publication
(10) Pub. No.: US 2025/0303008 A1
(43) Pub. Date: Oct. 2, 2025

(54) COMPONENT SANITIZER

(71) Applicant: Lenovo (United States) Inc., Morrisville, NC (US)

(72) Inventor: Cyan Godfrey, Chapel Hill, NC (US)

(21) Appl. No.: 18/618,699

(22) Filed: Mar. 27, 2024

(51) Int. Cl. A61L 2/10; A61L 2/26

(52) U.S. Cl. CPC A61L 2/10; A61L 2/26; A61L 2202/11; A61L 2202/122; A61L 2202/14

(57) ABSTRACT

One embodiment provides a device, including: a body including a top surface, the body being formed to create a cavity located under the top surface and within the body, wherein the cavity receives a component within the body; and a sanitation component located within the body and directed toward the cavity; wherein when the component is placed into the cavity, the sanitation component is activated. Other aspects are claimed and described.

### BACKGROUND

Many entities and companies have reduced the amount of office space that is available to employees. Rather than having a single desk or office space for a single employee, the entities and companies may have spaces that are utilized by multiple employees dependent upon which employee might be within the building at a particular time or day. This is more common with entities and companies allowing employees to work from home certain days of the week, entities and companies who have multiple shifts of employees who do not overlap, and/or the like. Additionally, companies and entities exist which provide work spaces for workers to utilize to perform work for another company. For example, such companies may provide a work space for a worker who works remotely for another company, but who wants to have an office space outside of the home. These work spaces that may be used by different people at different times may have some components or devices that are setup on the workspace for use by the employee or worker, for example, output devices (e.g., monitors, speakers, etc.), input devices (e.g., keyboards, touch screens, mice, etc.), and/or the like.

### BRIEF SUMMARY

In summary, one aspect provides a device, including: a body including a top surface, the body being formed to create a cavity located under the top surface and within the body, wherein the cavity receives a component within the body; and a sanitation component located within the body and directed toward the cavity; wherein when the component is placed into the cavity, the sanitation component is activated.

Another aspect provides a component sanitation device, including: a body including a top surface, a left side surface, a right side surface, a back surface, and a front surface, wherein the top surface, left side surface, right side surface, and back surface are formed to create a cavity within the body; wherein the front surface extends between a front portion of the left side surface and a front portion of the right side surface and extends downwards from the top surface, wherein the front surface, left side surface, and right side surface are formed to create an opening below the front surface on the front of the body to access the cavity; a sanitation component including an ultraviolet light located within the cavity; and a mechanism located at the top surface of the body and extending into the cavity through an opening in the top surface, wherein the mechanism pivots at a location of the mechanism located at the opening of the top surface of the body.

A further aspect provides a method for sanitizing a component, including: detecting, at a sanitation device, a component located within a cavity of the sanitation device, wherein the cavity includes a sanitation component directed toward the cavity, wherein the sanitation device includes a body including a top surface, the body being formed to create the cavity located under the top surface and within the body; and activating the sanitation component.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example of information handling device circuitry.

FIG. 2 illustrates another example of information handling device circuitry.

FIG. 3 illustrates an example method for activating a sanitation component upon detection of a component within a cavity of a sanitation device.

FIG. 4 illustrates an overall view of an exemplary sanitation device.

FIG. 5A illustrates an example first step of a pictorial progression of a component within a cavity of a sanitation device being sanitized and then ejected using a pivotable mechanism and an information handling device being propped using the pivotable mechanism.

FIG. 5B illustrates an example second step of a pictorial progression of a component within a cavity of a sanitation device being sanitized and then ejected using a pivotable mechanism and an information handling device being propped using the pivotable mechanism.

FIG. 5C illustrates an example third step of a pictorial progression of a component within a cavity of a sanitation device being sanitized and then ejected using a pivotable mechanism and an information handling device being propped using the pivotable mechanism.

FIG. 5D illustrates an example final step of a pictorial progression of a component within a cavity of a sanitation device being sanitized and then ejected using a pivotable mechanism and an information handling device being propped using the pivotable mechanism.

FIG. 6A illustrates an example sanitation device having ports.

FIG. 6B illustrates an example sanitation device having an indentation on the top surface.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

While many portable information handling devices (e.g., laptop computers, tablets, convertible devices, etc.) have all the components necessary for performing work, it is generally easier to perform substantial work using larger devices. For example, while a laptop computer has a keyboard, touch pad, and display, for performing work for substantial lengths of time, it is generally more efficient to perform the work using a full-sized keyboard, a mouse, and one or more displays. However, carrying these components into a work space would be very cumbersome. Additionally, having to set up some of these components would require additional time that could be utilized for actually performing work instead of setting up a work station. Thus, it is convenient for employees or workers to not have to carry the components of a work station in and out of a workspace.

However, by supplying the components of a workspace, this means that multiple people are touching and using the same components. Many people may not like using the same components that other people have touched due to a concern with spreading germs and/or disease. One solution is that people may carry the most commonly or frequently touched components with them, such as a keyboard and mouse. However, as previously mentioned, this causes extra components to be carried by the user and can become bulky and cumbersome. Additionally, if a person has to carry their own components, a user may choose a smaller version of the component due to size instead of one that may result in the most efficient performance of a task. Another solution is the person may carry sanitizing wipes that can be used to wipe down the components before use. However, after using the wipes, the user must wait for the component to dry before use. Additionally, wipes may not be effective at getting into grooves and spaces of components, thereby resulting in an incomplete sanitization of the device.

Accordingly, the described system and method provides a device for sanitizing components between uses. Generally, the described device will be utilized for sanitizing peripheral components of a computing device, for example, keyboard, mouse, other input devices, and/or the like. However, the described device could also be utilized to sanitize other components that can fit within the device, for example, tablets, projectors, cameras, smart phones, smart watches, gaming controllers, and/or the like. The sanitizing device includes a body having a top surface and being formed to create a cavity under the top surface within the body. The cavity is of a shape and size to receive the component to be sanitized. The cavity also includes a sanitation component, for example, an ultraviolet light, sanitizer or cleaning product mister, and/or the like, which is directed toward the cavity where the component will be located when the component is placed within the cavity. Once the component is placed within the cavity, the sanitation component is activated so as to sanitize the component.

The illustrated example embodiments will be best understood by reference to the figures. The following description is intended only by way of example, and simply illustrates certain example embodiments.

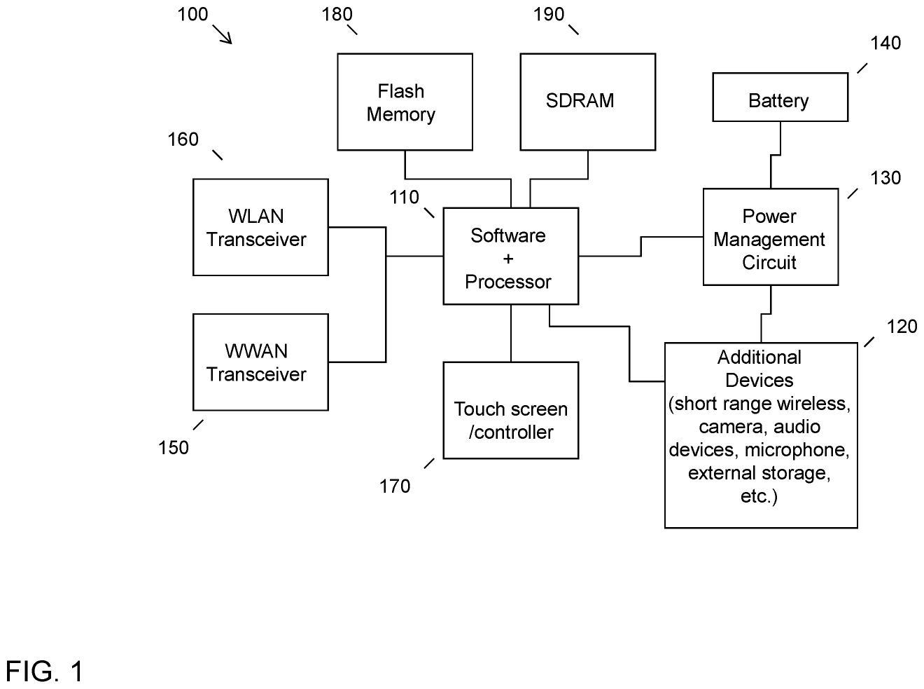

While various other circuits, circuitry or components may be utilized in information handling devices, with regard to smart phone and/or tablet circuitry 100, an example illustrated in FIG. 1 includes a system on a chip design found for example in tablet or other mobile computing platforms. Software and processor(s) are combined in a single chip 110. Processors comprise internal arithmetic units, registers, cache memory, busses, input/output (I/O) ports, etc., as is well known in the art. Internal busses and the like depend on different vendors, but essentially all the peripheral devices (120) may attach to a single chip 110. The circuitry 100 combines the processor, memory control, and I/O controller hub all into a single chip 110. Also, systems 100 of this type do not typically use serial advanced technology attachment (SATA) or peripheral component interconnect (PCI) or low pin count (LPC). Common interfaces, for example, include secure digital input/output (SDIO) and inter-integrated circuit (I2C).

There are power management chip(s) 130, e.g., a battery management unit, BMU, which manage power as supplied, for example, via a rechargeable battery 140, which may be recharged by a connection to a power source (not shown). In at least one design, a single chip, such as 110, is used to supply basic input/output system (BIOS) like functionality and dynamic random-access memory (DRAM) memory.

System 100 typically includes one or more of a wireless wide area network (WWAN) transceiver 150 and a wireless local area network (WLAN) transceiver 160 for connecting to various networks, such as telecommunications networks and wireless Internet devices, e.g., access points. Additionally, devices 120 are commonly included, e.g., a wireless communication device, external storage, etc. System 100 often includes a touch screen 170 for data input and display/rendering. System 100 also typically includes various memory devices, for example flash memory 180 and synchronous dynamic random-access memory (SDRAM) 190.

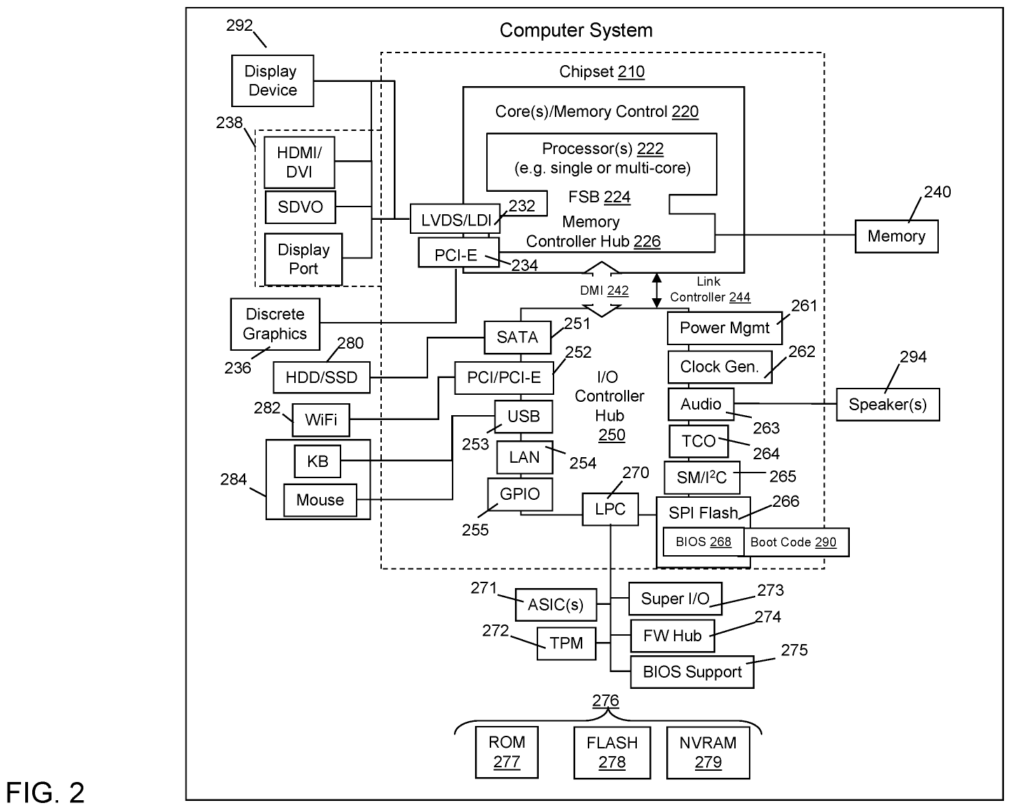

FIG. 2 depicts a block diagram of another example of information handling device circuits, circuitry, or components. The example depicted in FIG. 2 may correspond to computing systems such as personal computers, or other devices. As is apparent from the description herein, embodiments may include other features or only some of the features of the example illustrated in FIG. 2.

The example of FIG. 2 includes a so-called chipset 210 (a group of integrated circuits, or chips, that work together, chipsets) with an architecture that may vary depending on manufacturer. The architecture of the chipset 210 includes a core and memory control group 220 and an I/O controller hub 250 that exchanges information (for example, data, signals, commands, etc.) via a direct management interface (DMI) 242 or a link controller 244. In FIG. 2, the DMI 242 is a chip-to-chip interface (sometimes referred to as being a link between a “northbridge” and a “southbridge”). The core and memory control group 220 include one or more processors 222 (for example, single or multi-core) and a memory controller hub 226 that exchange information via a front side bus (FSB) 224; noting that components of the group 220 may be integrated in a chip that supplants the conventional “northbridge” style architecture. One or more processors 222 comprise internal arithmetic units, registers, cache memory, busses, I/O ports, etc., as is well known in the art.

In FIG. 2, the memory controller hub 226 interfaces with memory 240 (for example, to provide support for a type of random-access memory (RAM) that may be referred to as “system memory” or “memory”). The memory controller hub 226 further includes a low voltage differential signaling (LVDS) interface 232 for a display device 292 (for example, a cathode-ray tube (CRT), a flat panel, touch screen, etc.). A block 238 includes some technologies that may be supported via the low-voltage differential signaling (LVDS) interface 232 (for example, serial digital video, high-definition multimedia interface/digital visual interface (HDMI/DVI), display port). The memory controller hub 226 also includes a PCI-express interface (PCI-E) 234 that may support discrete graphics 236.

In FIG. 2, the I/O hub controller 250 includes a SATA interface 251 (for example, for hard-disc drives (HDDs), solid-state drives (SSDs), etc., 280), a PCI-E interface 252 (for example, for wireless connections 282), a universal serial bus (USB) interface 253 (for example, for devices 284 such as a digitizer, keyboard, mice, cameras, phones, microphones, storage, other connected devices, etc.), a network interface 254 (for example, local area network (LAN)), a general purpose I/O (GPIO) interface 255, a LPC interface 270 (for application-specific integrated circuit (ASICs) 271, a trusted platform module (TPM) 272, a super I/O 273, a firmware hub 274, BIOS support 275 as well as various types of memory 276 such as read-only memory (ROM) 277, Flash 278, and non-volatile RAM (NVRAM) 279), a power management interface 261, a clock generator interface 262, an audio interface 263 (for example, for speakers 294), a time controlled operations (TCO) interface 264, a system management bus interface 265, and serial peripheral interface (SPI) Flash 266, which can include BIOS 268 and boot code 290. The I/O hub controller 250 may include gigabit Ethernet support.

The system, upon power on, may be configured to execute boot code 290 for the BIOS 268, as stored within the SPI Flash 266, and thereafter processes data under the control of one or more operating systems and application software (for example, stored in system memory 240). An operating system may be stored in any of a variety of locations and accessed, for example, according to instructions of the BIOS 268. As described herein, a device may include fewer or more features than shown in the system of FIG. 2.

Information handling device circuitry, as for example outlined in FIG. 1 or FIG. 2, may be used in devices such as tablets, smart phones, personal computer devices generally, and/or electronic devices, which may be placed within the sanitation device for sanitization. Additionally, some of the circuitry described in connection with FIG. 1 and/or FIG. 2 may be utilized within the sanitation device for facilitating sanitization of a component. For example, the circuitry outlined in FIG. 1 may be implemented in a tablet or smart phone embodiment, whereas the circuitry outlined in FIG. 2 may be implemented in a personal computer embodiment.

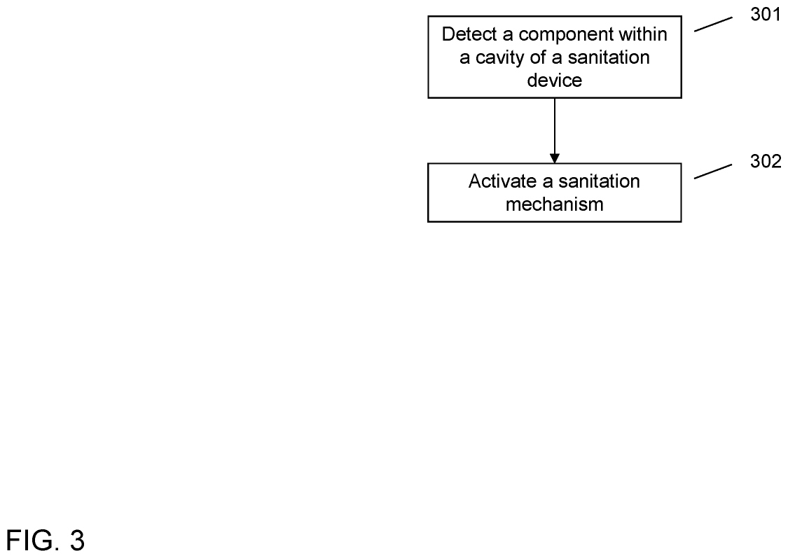

FIG. 3 illustrates an example method for performing sanitization of a component that is placed within a sanitation device. While the system may include known hardware and software components and/or hardware and software components developed in the future, the device itself is specifically designed to perform the functions as described herein to sanitize components placed within the sanitation device. Additionally, the sanitation device includes components, modules, and features that are unique to the described system. At 301, the system detects, at a sanitation device, a component located within a cavity of the sanitation device. The cavity includes a sanitation component that is directed toward the cavity. Upon detection of the component located within the cavity, the system activates the sanitation component at 302, thereby sanitizing the component located within the sanitation device.

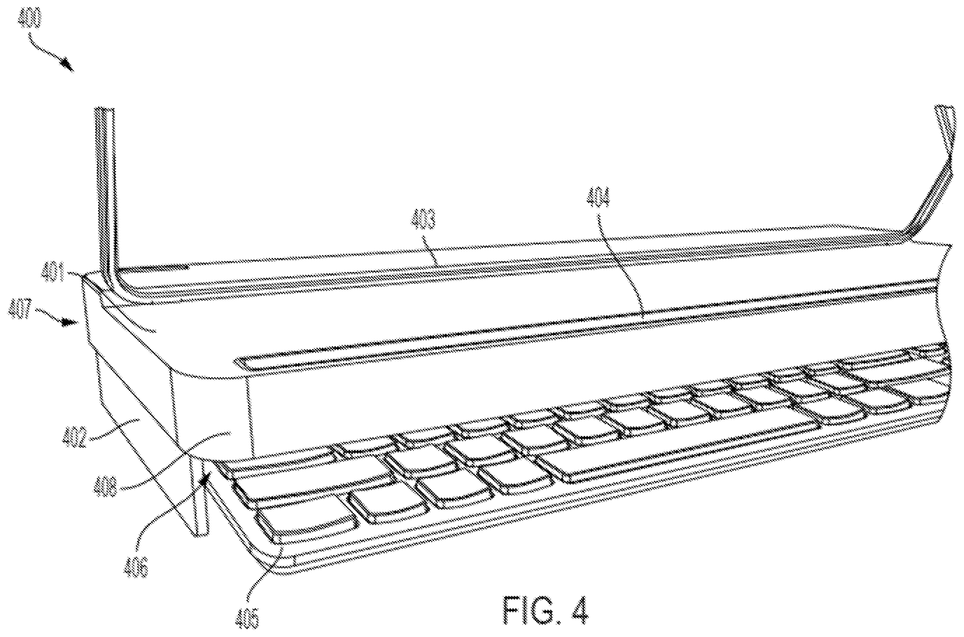

An example overall view of the sanitation device is illustrated in FIG. 4. While FIG. 4 illustrates an example, it should be understood that the shape and size of the sanitation device may vary based upon aesthetic desires, application or use cases, a component or components that will be used within the sanitation device, and/or the like. Thus, in the example, the sanitation device has an overall box-like shape within an internal cavity. However, the sanitation device could have any overall shape, for example, a cylindrical-like shape, a circular-like shape, an irregular shape, and/or the like.

The sanitation device includes a body 400 including a top surface 401. While shown as a relatively flat smooth top surface in FIG. 4, the top surface 401 may have a different surface profile, for example, a bumpy surface, an undulating surface, a surface having a texture, an irregular surface, a surface having angles, and/or the like. However, the top surface will generally extend as a horizontal surface, regardless of the overall shape of the top surface. The sanitation device is formed as to create a cavity 406 under the top surface and within the body 400. The cavity 406 is designed to receive a component 405 for sanitization.

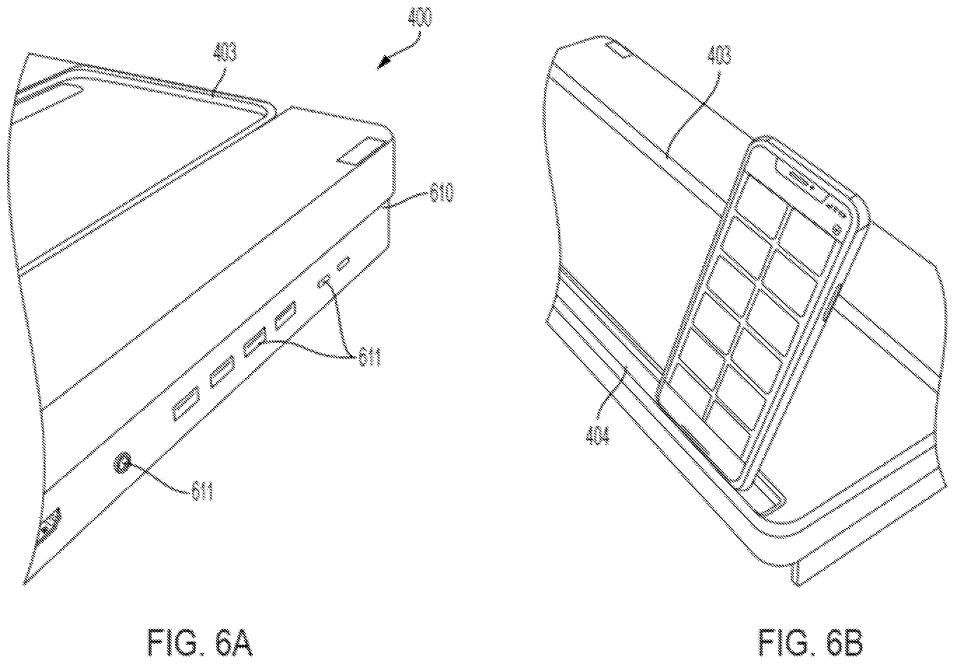

In order to form the cavity, the body 400 may include surfaces additional to the top surface 401. However, as previously mentioned, the number of surfaces may vary based upon the overall shape and size of the sanitation device. In the example illustrated in FIG. 4, the body 400 includes a left surface 402 and a right surface (not illustrated in FIG. 4, but essentially a mirrored version of the left surface 402). The right surface is visible in FIG. 6B. The left surface 402 and right surface are on a plane perpendicular to the top surface 401. In other words, the left surface 402 and right surface are formed to extend in a different direction from the top surface, thereby creating an overall three-dimensional object and shape. Like the top surface 401, the side surfaces may have different surface profiles. However, the side surfaces (e.g., left surface 402, right surface, back surface, front surface, etc.) generally extend as a vertical surface, regardless of the overall shape of the side surface. Even if the overall sanitation device body 400 were an egg shape, or circular shape, there will still be a substantially horizontal surface and one or more substantially vertical surfaces. The substantially horizontal surface on the top of the device will be referred to as the top surface and the substantially vertical surfaces will be referred to as side, back, or front surfaces.

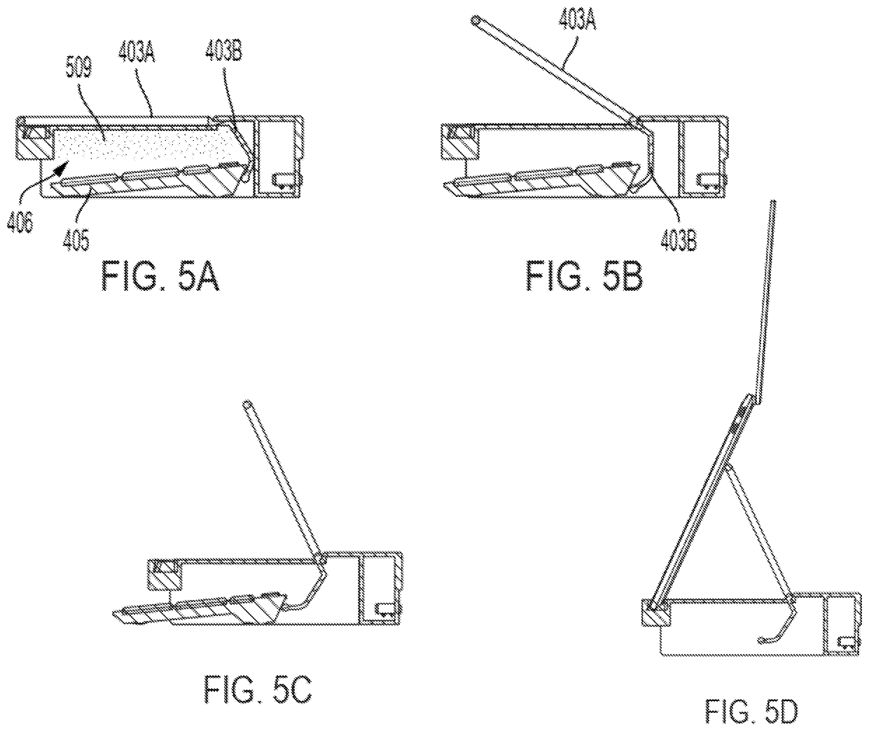

As illustrated in FIG. 4, the left surface 402 and the right surface, may not be single, flat surfaces that extend in a straight line from the top surface. Rather, the left surface 402 and the right surface may be formed as multiple pieces. As illustrated in FIG. 4, the top surface 401 and portions of the left surface 402 and right surface are formed as a single unitary piece, referred to as a top portion 407 of the sanitation device body 400. The left surface 402 and the right surface additionally include additional pieces that are fit within the top portion 407. These pieces will be referred to as legs of the left side and the right side of the sanitation device body 400. FIG. 5 illustrates a side view of the sanitation device and has a different shape for the legs and top portion.

Additionally, the sanitation device body 400 includes a back surface, not illustrated in FIG. 4, but visible in FIG. 6A. The back surface, like the left surface 402 and the right surface is on a plane perpendicular to the top surface 401. In other words, the back surface is formed to extend in a different direction from the top surface 401 and in the same direction from the top surface 401 as the left surface 402 and the right surface. The back surface may extend between the left surface 402 and the right surface and down from the top surface 401. Like the side surfaces, the back surface may not be a single piece, nor may it be a flat surface that extends in a straight line from the top surface. Rather, like the side surfaces, the back surface may be formed as multiple pieces.

The sanitation device body 400 also includes a front surface 408. Like the left surface 402, the right surface, and the back surface, the front surface 408 is on a plane perpendicular to the top surface 401. In other words, the front surface 408 is formed to extend in a different direction from the top surface 401 and in the same direction from the top surface 401 as the left surface 402, right surface, and back surface. However, instead of extending all the way to the bottom of the left surface 402, right surface, and back surface, the front surface 408 only extends partially down from the top surface 401 towards the bottom of the other side surfaces. This creates an opening in the front of the sanitation device body 400. The side surfaces and top surface 401 are formed together to create a cavity 406 within the body 400 of the sanitation device. Thus, the side surfaces form wall-type structures of the sanitation device body 400 and the top surface provides a cover or roof of the sanitation device body 400, thereby creating an internal cavity 406 of the sanitation device body 400.

The opening created by the front surface 408 in conjunction with the side surfaces allows for inserting or placing a component 405 within the cavity 406 of the sanitation device. As should be understood, the opening may be created in the front of the sanitation device body 400, one of the sides of the sanitation device body 400, the back of the sanitation device body 400, or even the top of the sanitation device body 400. Rather than requiring a specific location for the opening, the opening can be anywhere on the device that allows for a component 405 to be inserted into the sanitation device body 400. Additionally, the opening may be covered by a door, panel, or other mechanism that can be opened to allow the component 405 to be placed within the cavity 406 of the sanitation device and closed when the component 405 is within the cavity 406 or outside of the cavity 406. The size of the opening may be based upon the component or components that will be sanitized by the sanitation device. For example, a sanitation device that is designed to sanitize mice might have an opening that is narrower and taller than an opening needed for a keyboard. Alternatively, the opening may be of a size and shape that allows for the insertion of any component within the cavity of the sanitation device body 400.

While illustrated in FIG. 4 as the side surfaces (e.g., left side surface, right side surface, back surface, front surface, etc.) ending at the top surface 401, it should be noted that the surfaces could extend past the top surface, thereby making an edge around the top surface 401. Additionally, only a subset of the side surfaces may extend past the top surface 401. In other words, the height of the side surfaces as compared to the top surface do not have to be the same, nor do they all have to end at the top surface 401.

As an example sanitation device body 400, the top portion may include a top surface, a left-side surface, a right-side surface, a back surface, and a front surface. These surfaces may all be formed as a single unitary piece to make the top portion. The sanitation device may include additional left side surface pieces, right side surface pieces, and back surface pieces that fit into the top portion. Thus, like the left side surface and the right-side surface, the back surface may also have a leg piece. The legs may be all formed as a single unitary piece or may be separate pieces that are mechanically connected together, for example, using glue, epoxy, welds, fasteners (e.g., rivets, screws, bolts, etc.), complementary connectors (e.g., hooks and holes, complementary joining techniques, pins and holes, etc.), and/or the like.

As should be understood, in the case that the overall shape of the sanitation device is different than a box-like shape, the left surface, right surface, and back surface may not be perpendicular to the top surface, but may instead be in a different plane as the top surface but not a perpendicular plane. Additionally, the sanitation device body 400 may have additional surfaces. For example, if the sanitation device has a hexagonal shape, the sanitation device body 400 would include six perpendicular surfaces from the top surface. As another example, if the sanitation device body 400 had vertical surfaces that had a triangular shape, the left surface would not be in a straight perpendicular plane from the top surface.

Additionally, the size of the sanitation device may vary based upon the component that will be placed within the sanitation device body 400. For example, the example illustrated in the figures is designed for a mechanical keyboard. However, other components that may be sanitized using the sanitation device include, but is not limited to, mice, tablets, smart phones, smart watches, speakers, headphones, microphones, projectors, and/or the like. Thus, the sanitation device may be of a size and shape that allows for the sanitization of those components. Additionally, the sanitation device may be designed to allow for the sanitization of multiple components at the same time. For example, the device may be sized to sanitize both a keyboard and mouse at the same time. As another example, the device may be sized to sanitize multiple keyboards at the same time.

The sanitation device body 400 may or may not have a bottom surface. If the sanitation device body 400 has a bottom surface, the bottom surface may be near the bottom of the side surfaces and run in a perpendicular plane as compared to the side surfaces. As with the top surface, the bottom surface may not be a flat surface that is perfectly perpendicular to the side surfaces, particularly if the side surfaces are not straight vertical surfaces. However, the bottom surface runs in a general horizontal direction that is relatively parallel to the top surface and generally perpendicular to the general vertical direction of the side surfaces. The bottom surface, if included, provides a surface that the component, when within the cavity of the sanitation device, sits upon.

The bottom surface, if included, may be the same size as the top surface, or may be a different size. In other words, the bottom surface may be recessed as compared to the front surface, may extend to the front surface, may extend past the front surface, and/or the like. The bottom surface, if included, may extend between the left surface and the right surface. While connected to the left surface and the right surface, the bottom surface may extend past the left surface and the right surface, thereby creating a ledge past the left surface and the right surface. Additionally, the bottom surface may extend to the back surface. Like the left surface and the right surface, the bottom surface may also extend past the back surface. However, unlike the left surface and the right surface, the bottom surface may not extend all the way to the back surface, thereby creating a hole between the bottom surface and the back surface.

Alternatively, the sanitation device body 400 may not have a bottom surface. The sanitation device may sit on a secondary surface (e.g., desk, table, cabinet, horizontal surface, etc.) and that surface may act as a bottom surface for the sanitation device body 400. In other words, the component 405, when within the cavity 406 of the sanitation device body 400, may sit on the secondary surface and be otherwise enclosed by the sanitation device body 400.

The sanitation device may include an indentation 404 on the top surface 401. The indentation 404 may be any size, shape, and/or depth. In the example illustrated in the figures, the indentation 404 extends through the top surface 401, thereby making a hole or slot in the top surface 401. The indentation 404 may provide a notch, recess, slot, or other indentation for holding a device on the top of the sanitation device. Utilization of the indentation 404 for holding a device is illustrated in FIG. 6B. As shown in FIG. 6B, the indentation 404 is of a size that allows for the smart phone to be inserted into the indentation and prop the smart phone in a viewable and/or usable manner. As another example, illustrated in FIG. 5D, a laptop is inserted in the indentation and is being propped using the indentation in conjunction with a pivotable mechanism 403. This allows the laptop to be propped up in a manner that allows a user to look ahead at the display instead of down at the display as when the laptop is used on a flat surface. Thus, even when not in use as a sanitation device, the sanitation device provides functionality to perform other tasks such as propping other devices or components. Accordingly, the indentation may be of a size and shape that allows for the propping of devices or components.

The sanitation device includes a pivotable mechanism 403 on the top surface 401. While illustrated in FIG. 4, the pivotable mechanism 403 is best seen in FIGS. 5A-5D. In the example illustrated in the figures, the top surface portion of the pivotable mechanism 403A is a metal frame having an internal opening. However, the top surface portion of the pivotable mechanism 403A may be a solid piece of material (e.g., metal, plastic, wood, combination of materials, etc.) having no internal opening. Alternatively, the top surface portion of the pivotable mechanism 403A may have a design that includes solid portions and open portions. Additionally, the top surface pivotable mechanism 403A may have designs (e.g., graphics, art, logos, trademarks, information, directions, etc.) etched, painted, stained, or otherwise included on the top surface portion of the pivotable mechanism 403A either on the top of the mechanism, the bottom of the mechanism, both the top and bottom, and/or the like. The top surface portion 403A may be utilized to prop devices, for example, as shown in FIG. 5D. The top surface portion 403A may be used in conjunction with the indentation 404 to hold a device in an upright position. To facilitate this propping, the top surface portion 403A may pivot upwards from the top surface 401 to a predetermined location or position. A device can then be lain against the top surface portion 403A and within the indentation 404 and held at a predetermined location on the body 400 of the sanitation device.

The pivotable mechanism 403 has a portion 403A located on the top surface 401 and a portion 403B located within the cavity 406 of the body 400 of the sanitation device. Thus, the top surface 401 includes one or more holes/slots that allows the pivotable mechanism to extend between the top surface 401 and the cavity 406. Depending on the design of the pivotable mechanism 403, a single hole, multiple holes, slots, and/or the like, may be utilized to extend the pivotable mechanism 403 between the top surface 401 and the cavity 406. The pivotable mechanism 403 may pivot at a location of the pivotable mechanism 403 that is located at the opening of the top surface 401 of the body. In other words, where the pivotable mechanism 403 breaches the top surface 401 may be a pivot point for the pivotable mechanism 403. Thus, when the pivotable mechanism 403 is moved with respect to the top surface, the pivotable mechanism 403 pivots about the pivot point and the cavity portion 403B also moves.

The cavity portion 403B extends into the cavity 406 towards a back portion of the cavity 406, as illustrated in FIG. 5A. Particularly, the cavity portion 403B is utilized to push a component 405 that is within the cavity 406 towards the front of the cavity 406 and out of the body 400 of the sanitation device. Thus, the cavity portion 403B is located within the cavity such that when the pivotable mechanism 403 is in a closed position (i.e., the top surface portion 403A is down towards the top surface 401), the cavity portion 403B will be behind (with respect to the front surface 408 of the body 400) any component 405 within the cavity 406. Additionally, the cavity portion 403B may be located so that when the pivotable mechanism 403 is moved towards an open position (i.e., the top surface portion 403A is moved away from the top surface 401), the cavity portion 403B touches and moves any component 405 within the cavity 406. The cavity portion 403B may be of any shape and size and may be dependent upon the component that the sanitation device is designed to sanitize. For example, the cavity portion 403B may include a push bar that extends a majority of the width of the sanitation device, may be one or more push rods or push handles, and/or the like.

An illustration of the pivotable mechanism 403 pivoting and pushing a component 405 out of the cavity 406 is illustrated in FIG. 5A-FIG. 5D. In FIG. 5A, a component 405 is located within the cavity 406 of the body 400 of the sanitation device. This illustration shows the pivotable mechanism 403 in a closed position. As seen in this illustration, cavity portion 403B is located behind the component 405 towards the back of the cavity 406. In this illustration, cavity portion 403B is touching component 405. However, this is not strictly necessary. The top surface portion 403A is located on the top surface 401. In this illustration, the top surface portion 403A is touching the top surface 401. However, this is not strictly necessary. In FIG. 5B, the pivotable mechanism 403 is being moved towards an open position. The top surface portion 403A is being moved away from the top surface 401. At the same time, the cavity portion 403B is moving towards a front portion of the cavity 406 and pushing the component 405 out of the cavity 406 towards the front of the body 400 of the sanitation device.

In FIG. 5C, the pivotable mechanism 403 is at an open position. The top surface portion 403A is in a position to make a prop bar for a device. The cavity portion 403B has moved forward within the cavity 406 and pushed the component 405 past the front surface 408 of the body 400 of the sanitation device. Thus, a user can touch and grab the component 405 to position the component 405 where the user wants to use it. FIG. 5D illustrates how the top surface portion 403A in an open position and the indentation 404 can be used in conjunction with each other to hold a device, in this example, a laptop computer, in an upright position.

The sanitation device includes a sanitation component located within the body 400 of the sanitation device. The sanitation component 509 is illustrated in FIG. 5A. The sanitation component 509 can be located anywhere within the body 400 of the sanitation device, but will be directed towards the cavity 406. In other words, the sanitation component 509 is located within the body 400 of the sanitation device such that it is directed towards a component 405 when the component 405 is located within the cavity 406. In the illustration of FIG. 5A, the sanitation component 509 is located towards the upper front portion of the cavity 406. However, this is not strictly necessary and the sanitation component 509 can be located in the upper middle of the cavity 406, towards the upper back of the cavity 406, at the back of the cavity 406, and/or the like. Additionally, the sanitation device may have more than one sanitation component 509.

The sanitation component 509 may be an ultraviolet light, a sanitizing spray or mist, or other sanitization device or technique that is currently available or may be available in the future. When the component 405 is within the cavity 406 the sanitation component(s) 509 may be activated, thereby sanitizing the component(s) 405 within the cavity 406. The sanitation component(s) 509 may run for a predetermined interval of time. For example, an ultraviolet light may be programmed to be active for a certain length of time, even if the component 405 is within the cavity 406 for a longer period of time.

In order to effectuate, the sanitization of the component(s) 405, the sanitation component may be activated once the component is placed within the cavity. It should be noted that the sanitation component does not have to be activated immediately after the component is placed within the cavity, but may be. One way to activate the sanitation component is to include a sensor on the sanitation device that can detect the presence of a component within the cavity or body of the sanitation device. The sensor may be located around the front opening, within the cavity, and/or the like. Such a sensor may include a line-of-sight sensor that activates when a line-of-sight between two components of the sensor is blocked (like a garage door sensor), a presence sensor, a proximity sensor, and/or any other type of sensor that can be used to detect a presence of a component or the placement of a component within the cavity. Upon detecting the presence of the component within the cavity, the sanitation component may be activated, either immediately or after a predetermined length of time.

Another technique for activating the sanitation component may be including a button, switch, motion activation switch, audible activation mechanism, gesture activation mechanism, or other activation mechanism on the sanitation device. A user can provide input to this activation mechanism (e.g., pushing the button, switching the switch, waving a hand over the switch, providing audible input to start the device, providing a gesture to start the device, etc.), thereby activating the sanitation component. This allows a user to start the sanitation device when desired.

The sanitation device may include lights or indicators that indicate the component(s) 405 is being sanitized or that the sanitation component(s) 509 is active. For example, during the active time of the sanitation component(s) 509, a light or other indicator may be lit indicating that the sanitation component(s) 509 is active. In the case that a sanitizing mist or spray is used, the light may remain lit until the component 405 is expected to be dry. Upon deactivation of the sanitation component(s) 509, for example, after the sanitation component(s) 509 have completed a sanitation cycle, the lights or indicators may turn off or change states to indicate that the component 405 has been sanitized and is ready to be removed from the sanitation device. For example, the sanitation device may have multiple lights or indicators, with one light or indicator dedicated to indicating the sanitation component(s) 509 is currently active, another light or indicator dedicated to indicating the component 405 has been sanitized and is ready to be removed, another light or indicator dedicated to indicating the sanitation device is ready to be used. Alternatively, the sanitation device may include a single light or indicator that changes states (e.g., different colors, different light patterns (e.g., flashing, solid, flash patterns, etc.), etc.) based upon a status of the sanitation device (e.g., ready for sanitizing, active sanitizing, sanitizing complete, component removed, component within the cavity, etc.). The lights or indicators may include any type of light or indicator, including, but not limited to, lights, light emitting diodes, display screens, audible output, haptic output, and/or the like.

As illustrated in FIG. 6A, the back surface 610 of the sanitation device body 400 may include ports 611 that allows for the connection of devices to the sanitation device. Thus, the sanitation device may act as a docking station in addition to a sanitation device. The user can plug different devices into the ports 611 that may allow for charging devices, connecting peripheral devices to a device (e.g., connecting monitors to a device docked on the sanitation device, connecting printers to a device docked on the sanitation device, etc.), connecting devices together using the sanitation device as an intermediary device, and/or the like. For example, a user may be able to plug a laptop device into the sanitation device and also plug a printer into the sanitation device, thereby allowing the two devices to communicate with each other through the sanitation device. The sanitation device may be powered utilizing any type of power source, for example, battery, wired power, solar power, activity-based power, and/or the like, or a combination thereof. Accordingly, the sanitation device may include components required for powering to the sanitation device. Thus, the ports 611 may include ports for connecting peripheral devices to the body of the sanitation device, ports for providing power to a device, ports for providing data transmission to, from, or between devices, ports for providing power to the sanitation device, ports for charging a battery of the sanitation device, and/or the like.

As an overall non-limiting example of the described system, a company may have desks and workstations that are not assigned to a particular individual. As employees enter the building, they may choose any open workstation. At the workstation is a set of monitors, a keyboard, a mouse, and sanitation device that also acts as a docking station for a laptop computer that is carried by the employee. The employee chooses a workstation and moves the pivotable mechanism of the sanitation device to an open position, which pushes a sanitized keyboard and mouse out of the sanitation device. Once the pivotable mechanism is in the open position, the user can position the laptop on the sanitation device to be propped up by the pivotable mechanism in conjunction with the indentation. The user can then connect any cables to the laptop computer to connect the laptop to peripheral devices, such as printers, network drives, the monitors, and/or the like. While in the open position, the user cannot put the keyboard and/or mouse back in the sanitation device, but could put a smaller device, such as a smart phone within the device and start a sanitation cycle to sanitize the smart phone.

Once the employee is ready to leave, the employee disconnects the laptop and removes it from the top of the sanitation device. The employee moves the pivotable mechanism to the closed position and places the keyboard and/or mouse into the opening of the sanitation device into the cavity. Upon detecting the keyboard and/or mouse within the cavity or detecting a user input indicating the sanitation cycle should begin, the sanitation device starts the sanitation cycle by activating the sanitation mechanism. In this example, the sanitation mechanism will be an ultraviolet light. Thus, the ultraviolet light is activated and sanitizes the keyboard and/or mouse, thereby making it ready for the next employee who chooses that workstation. The sanitation cycle runs for the prescribed length of time and the sanitation device then provides an indicator that the keyboard and/or mouse are now sanitized.

As will be appreciated by one skilled in the art, various aspects may be embodied as a system, method, or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random-access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Additionally, the term “non-transitory” includes all media except signal media.

Program code embodied on a storage medium may be transmitted using any appropriate medium, including but not limited to wireless, wireline, optical fiber cable, radio frequency, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices, and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A device, comprising:
a body comprising a top surface, the body being formed to create a cavity located under the top surface and within the body, wherein the cavity receives a component within the body; and
a sanitation component located within the body and directed toward the cavity;
wherein when the component is placed into the cavity, the sanitation component is activated.

2. The device of claim 1, comprising a pivotable mechanism located at the top surface of the body and extending into the cavity through an opening in the top surface, wherein the pivotable mechanism pivots at a location of the pivotable mechanism located at the opening of the top surface of the body.

3. The device of claim 2, wherein the portion of the pivotable mechanism extending into the cavity is located towards a back portion of the body and when the pivotable mechanism is pivoted upwards from the top surface of the body, the portion of the pivotable mechanism extending into the cavity moves towards a front portion of the body.

4. The device of claim 2, wherein the pivotable mechanism pivots upwards from the top surface to a position to hold an information handling device at a predetermined location on the body.

5. The device of claim 1, wherein the body comprises at least one port for connecting a peripheral device to the body.

6. The device of claim 1, wherein the body comprises at least one port providing power to a device.

7. The device of claim 1, wherein the top surface comprises an indentation.

8. The device of claim 1, comprises a front surface and wherein the front surface comprises an opening, wherein the cavity is accessible via the opening and the component is inserted through the opening into the cavity.

9. The device of claim 1, comprising a sensor for detecting a presence of the component within the body and, wherein the sanitation component is activated based upon the detected presence.

10. The device of claim 1, wherein the body comprises at least one activation mechanism, wherein input provided at the at least one activation mechanism activates the sanitation component.

11. The device of claim 1, wherein the component comprises at least one of: a keyboard and a mouse.

12. A component sanitation device, comprising:
a body comprising a top surface, a left side surface, a right side surface, a back surface, and a front surface, wherein the top surface, left side surface, right side surface, and back surface are formed to create a cavity within the body;
wherein the front surface extends between a front portion of the left side surface and a front portion of the right side surface and extends downwards from the top surface, wherein the front surface, left side surface, and right side surface are formed to create an opening below the front surface on the front of the body to access the cavity;
a sanitation component comprising an ultraviolet light located within the cavity; and
a mechanism located at the top surface of the body and extending into the cavity through an opening in the top surface, wherein the mechanism pivots at a location of the mechanism located at the opening of the top surface of the body.

13. The component sanitation device of claim 12, comprising an indentation located on the top surface.

14. The component sanitation device of claim 12, wherein the portion of the mechanism extending into the cavity is located towards a back portion of the cavity and when the mechanism is pivoted upwards from the top surface of the body, the portion of the mechanism extending into the cavity moves towards a front portion of the body.

15. The component sanitation device of claim 12, wherein the body comprises at least one port for connecting a peripheral device to the body.

16. The component sanitation device of claim 12, wherein the body comprises at least one port providing power to a device.

17. The component sanitation device of claim 12, comprising a sensor for detecting a presence of a component within the cavity and, wherein the ultraviolet light is activated based upon the detected presence.

18. The component sanitation device of claim 12, wherein the body comprises at least one activation mechanism, wherein input provided at the at least one activation mechanism activates the ultraviolet light.

19. The component sanitation device of claim 12, wherein the mechanism pivots upwards from the top surface to a position to hold an information handling device at a predetermined location on the body.

20. A method for sanitizing a component, comprising:
detecting, at a sanitation device, a component located within a cavity of the sanitation device, wherein the cavity comprises a sanitation component directed toward the cavity, wherein the sanitation device comprises a body comprising a top surface, the body being formed to create the cavity located under the top surface and within the body; and
activating the sanitation component.
